(19) United States
(12) Patent Application Publication
(10) Pub. No.: US 2025/0189499 A1
(43) Pub. Date: Jun. 12, 2025

(54) CHROMIUM-FREE HALIDES DETERMINATION IN DRILLING FLUIDS

(71) Applicant: Halliburton Energy Services, Inc., Houston, TX (US)

(72) Inventors: Preston Andrew MAY, Houston, TX (US); Jay DEVILLE, Houston, TX (US)

(73) Assignee: Halliburton Energy Services, Inc., Houston, TX (US)

(21) Appl. No.: 18/533,152

(22) Filed: Dec. 7, 2023

(51) Int. Cl. G01N 31/02; E21B 21/06; G01N 1/40; G01N 33/28

(52) U.S. Cl. CPC G01N 31/02; E21B 21/065; G01N 1/4077; G01N 33/2823

(57) ABSTRACT

Apparatus for determining an amount of halide in a drilling fluid and methods of use. An example apparatus utilizes an analyzer configured to determine an amount of a halide precipitate in a substantially solids-free sample of a drilling fluid by measuring the amount of the precipitated halide produced upon contacting the substantially solids-free sample of drilling fluid with a cation. A solids removal device is configured to prepare the substantially solids-free sample by removing solids from a sample of the drilling fluid to produce the substantially solids-free sample fluid.

### TECHNICAL FIELD

The present disclosure relates generally to wellbore operations, and more particularly, to the use of a turbidimeter to measure the halide precipitates in a drilling fluid sample in order to determine the halide concentration in the drilling fluid without the use of chromium compounds or titration techniques.

### BACKGROUND

For some wellbore operations, it may be desirable to measure the halide concentration of the drilling fluid. Some halides may be used for lubricating the drill bit and improving the ability of the drilling fluid to carry cuttings. In some drilling operations, an increase in the halide concentration beyond the base level may indicate that the drilling operation has encountered a saline formation or even a water reservoir. Additionally, the concentration of halides may need to be monitored and regulated in formations with high clay content to manage clay swelling that could potentially damage a subterranean formation.

The halide concentration in a drilling fluid may be measured by titration analysis using chromate indicator solutions. Titration analysis relies on a visual indication which may be subjective and difficult to reproduce. Additionally, chromates may not be available in some locations and/or may be a potential hazard to the handling wellsite personnel.

The measurement of the halide concentration in a drilling fluid is an important part of a wellbore operation. The present invention provides improved apparatus and methods for the measurement of the halide concentration of the drilling fluid without the use of chromium compounds or titration techniques.

### BRIEF DESCRIPTION OF THE DRAWINGS

Illustrative examples of the present disclosure are described in detail below with reference to the attached drawing figures, which are incorporated by reference herein, and wherein:

FIG. 1 is a schematic illustrating an example drilling system utilizing a drilling fluid in accordance with one or more examples described herein;

FIG. 2 is a diagram illustrating a method of measuring the concentration of a halide in a drilling fluid sample in accordance with one or more examples described herein;

FIG. 3 is a schematic illustrating a system for determining the halide concentration in a drilling fluid sample in accordance with one or more examples described herein;

FIG. 4 is a schematic illustrating the use of a turbidimeter in accordance with one or more examples described herein;

FIG. 5 is a schematic continuing the illustrated use of a turbidimeter in accordance with one or more examples described herein; and

FIG. 6 is a graph showing turbidimetry results in accordance with one or more examples described herein.

The illustrated figures are only exemplary and are not intended to assert or imply any limitation with regard to the environment, architecture, design, or process in which different examples may be implemented.

### DETAILED DESCRIPTION

The present disclosure relates generally to wellbore operations, and more particularly, to the use of a turbidimeter to measure the halide precipitates in a drilling fluid sample in order to determine the halide concentration in the drilling fluids without the use of chromium compounds or titration techniques.

In the following detailed description of several illustrative examples, reference is made to the accompanying drawings that form a part hereof, and in which is shown by way of illustration specific examples that may be practiced. These examples are described in sufficient detail to enable those skilled in the art to practice them, and it is to be understood that other examples may be utilized, and that logical structural, mechanical, electrical, and chemical changes may be made without departing from the spirit or scope of the disclosed examples. To avoid detail not necessary to enable those skilled in the art to practice the examples described herein, the description may omit certain information known to those skilled in the art. The following detailed description is, therefore, not to be taken in a limiting sense, and the scope of the illustrative examples are defined only by the appended claims.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the examples of the present invention. At the very least, and not as an attempt to limit the application of the doctrine of equivalents to the scope of the claim, each numerical parameter should at least be construed in light of the number of reported significant digits and by applying ordinary rounding techniques. It should be noted that when “about” is at the beginning of a numerical list, “about” modifies each number of the numerical list. Further, in some numerical listings of ranges some lower limits listed may be greater than some upper limits listed. One skilled in the art will recognize that the selected subset will require the selection of an upper limit in excess of the selected lower limit.

In the following discussion and in the claims, the terms “including” and “comprising” are used in an open-ended fashion, and thus should be interpreted to mean “including, but not limited to.” Unless otherwise indicated, as used throughout this document, “or” does not require mutual exclusivity.

The terms uphole and downhole may be used to refer to the location of various components relative to the bottom or end of a well. For example, a first component described as uphole from a second component may be further away from the end of the well than the second component. Similarly, a first component described as being downhole from a second component may be located closer to the end of the well than the second component.

The terms upstream and downstream may be used to refer to the location of various components relative to one another in regards to the flow of a sample through said components. For example, a first component described as upstream from a second component will encounter a sample before the downstream second component encounters the sample. Similarly, a first component described as being downstream from a second component will encounter the sample after the upstream second component encounters the sample.

The present disclosure relates generally to wellbore operations, and more particularly, to the use of a turbidimeter to measure the halide precipitates in a drilling fluid sample in order to determine the halide concentration in the drilling fluids without the use of chromium compounds or titration techniques. Advantageously, the methods and systems disclosed herein may be used to determine the halide concentration of a drilling fluid. Moreover, the determination of the halide concentration may be made without reliance on titration or the use of chromium compounds to form the indicator solution necessary for titration analysis. Advantageously, gravimetric and/or volumetric analysis may be used in addition to or in place of turbidimetry. The methods herein utilize cations to form insoluble halide salts that will precipitate out of the sample of the drilling fluid. The concentration of the precipitated salts may then be determined and this value may be extrapolated to determine the concentration of the halides within the drilling fluid. An additional advantage is that the disclosed methods and systems may be used on both aqueous and non-aqueous drilling fluids.

The methods disclosed herein include obtaining a sample of the drilling fluid. The sample of the drilling fluid should be obtained from a circulated drilling fluid so as to measure the volume of halides potentially entering the drilling fluid from the formation and/or the loss of halides to the formation. After the drilling fluid sample is obtained, the solids within the drilling fluid sample are removed to produce a substantially solids-free fluid. As used here, “substantially” refers to a fluid where 90% or more of the solids, by weight of the total solids in the sample, have been removed. A soluble salt may be added to the drilling fluid to dissociate into its respective ions. The cation from the dissociated salt may then contact a halide anion to produce a precipitate and form a test solution to be analyzed. An analyzer may be used to measure the concentration of precipitated halide from the drilling fluid sample. The data may be extrapolated to determine the concentration of halides in the drilling fluid. In some examples, measuring the amount of the precipitate may be performed by turbidity analysis of the test solution, by gravimetric weight analysis of the precipitate, or by volumetric analysis of the precipitate.

As used herein, the term “halides” includes fluorides, chlorides, iodides, and bromides. The halides within the drilling fluid may be sodium halides, potassium halides, calcium halides, magnesium halides, zinc halides, ammonium halides, tetramethylammonium halides, cesium halides, strontium halides, choline halides, or any combination thereof. These halides may be naturally occurring within the subterranean formation or may be added to the drilling fluid to enhance a property of the drilling fluid. In some examples, the halides may be added to the drilling fluid to treat a zone of the subterranean formation, such as a wellbore zone comprising reactive clay. Through periodic monitoring, the concentration of the halide can be optimized and maintained at a target range to reduce or eliminate potential issues from occurring with the drilling operation. Described herein are methods, both qualitative and quantitative, that can determine a concentration of halides in a drilling fluid based on selective precipitation.

Various insoluble halide salts may be formed through the addition of select soluble salts to the drilling fluid which may dissociate and make available a cation for ion exchange with the dissolved halides. A halide precipitate is formed and this precipitate may be measured to determine the concentration of the halide in the sample.

The drilling fluid may be an aqueous-based or non-aqueous-based fluid and comprise one or more of water, brines, or hydrocarbons. The water may be fresh water, seawater, or salt water, for example. The hydrocarbon fluid may be mineral oils, biodegradable esters, olefins, or any other oleaginous material or variant thereof.

The sample of drilling fluid is processed to remove solids from the sample thereby producing a substantially solids-free fluid. The substantially solids-free fluid is then contacted with a cation to produce a precipitate in a test solution. The precipitate may be formed from a precipitated halide, for example, silver halide. The solids can be removed from the drilling fluid sample by filtration to produce a filtrate. In other examples, the solids can be removed by centrifugation, simple settling, or dissolution. If the drilling fluid is a non-aqueous-based fluid, the fluid sample may be diluted before and/or after filtration. The non-aqueous-based drilling fluid sample may be diluted with a solution of water and certain organic solvents such as, glycol ethers and derivatives thereof. Examples of the glycol ethers may include, but are not limited to, propylene glycol n-propyl ether, ethylene glycol monomethyl ether, ethylene glycol monoethyl ether, ethylene glycol monopropyl ether, ethylene glycol monoisopropyl ether, ethylene glycol monobutyl ether, propylene glycol monomethyl ether, diethylene glycol monomethyl ether, diethylene glycol monoethyl ether, diethylene glycol n-butyl ether, ethylene glycol, monoethyl ether acetate, propylene glycol methyl ether acetate, or any combination thereof. The organic solvent should not form stable ligands with the cation of the halide. This dilution process may occur before and/or after the solids are removed from the drilling fluid sample. Filtration of the drilling fluid sample may be performed by any sufficient method. One preferred method is pressure filtration, where the drilling fluid sample is placed in a vessel and a gas is introduced at high pressure to force the fluid components of the drilling fluid sample through a filter paper or screen to provide the substantially solids-free sample. An example of a standard filtration test is API 13B-1 from the American Petroleum Institute's recommended practices.

A volume of cation is added to the substantially solids-free sample to produce the precipitate. The cation is added through the addition of metal salts to the substantially solids-free sample which will then undergo ion exchange to form the insoluble halide precipitate. Any metal salt sufficient for precipitate formation may be used, including, but not limited to, those metal salts containing the cations of Ag⁺, Pb²⁺, Hg²⁺, Cu⁺, or any combination thereof. The amount of cation to be added is an amount known to be in excess of the halide present in the drilling fluid. The excess cation will form a maximum amount of precipitate in the test solution. The test solution may be diluted prior to determining the amount of precipitate formed if desired.

The test solution is the fluid suspension containing the halide precipitate. The test solution can be analyzed to determine the amount of precipitate formed. In some examples, turbidimetry is performed on the test solution. The test solution is analyzed using a turbidity meter to measure the amount of precipitate in the test solution and determine the concentration of halides in the sample of drilling fluid. From this data, the concentration of halides in the drilling fluid may then be estimated. In some alternative examples, gravimetric analysis is performed on the test solution. The test solution may be filtered and the precipitate dried and weighed to measure the amount of precipitate in the test solution and determine the amount of halides present in the sample of drilling fluid. From this data, the concentration of halides in the drilling fluid may then be estimated. In other alternative examples, volumetric analysis is performed on the test solution. The precipitate is allowed to settle in a graduated vessel and the volume of precipitate is measured to determine the concentration of halides in the sample of drilling fluid. From this data, the concentration of halides in the drilling fluid may then be estimated.

In some examples, the sample of drilling fluid is diluted prior to testing. This dilution may be separate from or in addition to the dilution that may be performed on non-aqueous-based drilling fluid samples. The substantially solids-free fluid may be diluted prior to contact with the cation. The test solution may be diluted prior to measuring the amount of precipitate formed. In some examples, the sample of drilling fluid may be concentrated prior to testing. The substantially solids-free fluid may be stripped prior to measuring the amount of precipitate formed. The concentration of precipitate in the test solution may be optimized for the method used for determining the amount of halide present in the sample of drilling fluid.

A concentration of halide in the drilling fluid may be adjusted based on the measured concentration of the halide precipitate in the test solution. For example, the concentration of halide in the drilling fluid may be increased if the test solution indicates that the measured amount of halide is below a threshold concentration. In another example, the concentration of halide in the drilling fluid may be decreased if the test solution indicates that the measured amount of halide is above a threshold concentration. The methods described herein may be performed in the field at the site of the wellbore. The methods described herein may be automated. In certain examples, the methods are performed by equipment without human interaction.

Optionally, the step of removing solids from the sample may include steps to remove potentially interfering cations. The step of removing solids from the sample may include removing solids from the sample to produce an initial substantially solids-free fluid, contacting the initial substantially solids-free fluid with a compound to produce an initial precipitate in an initial test solution, and removing the initial precipitate from the initial test solution to produce the substantially solids-free fluid for further analysis. Optionally, the initial substantially solids-free fluid may be contacted with a compound or compounds to remove potentially interfering cations and to produce an initial precipitate in an initial test solution. These initial solids may be removed by filtration, centrifugation, simple settling, or dissolution. The compounds added will be determined based on what interfering cations are known or expected to be in the substantially solids-free fluid. This process is optional and may not conducted if interfering cations are not present or are expected to not be present.

Optionally, the pH of the test solution may be adjusted to about 2 to about 13. The pH may be adjusted to improve precipitation of the halide. In some examples, the pH may be adjusted by adding hydrochloric acid or an acid known to those skilled in the art to the test solution. In some alternative examples, the pH may need to be decreased. The pH may be adjusted by adding sodium hydroxide or a base known to those skilled in the art to the test solution.

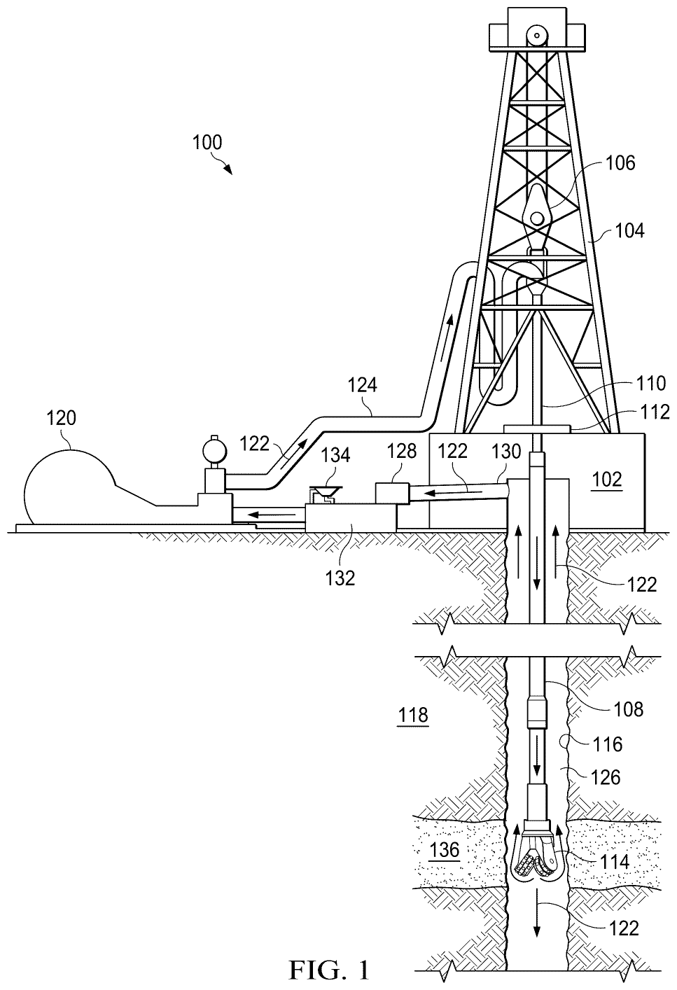

FIG. 1 illustrates a schematic of a drilling assembly 100 in which a drilling fluid 122 as disclosed above may be used. It should be noted that while FIG. 1 generally depicts a land-based drilling assembly, those skilled in the art will readily recognize that the principles described herein are equally applicable to subsea drilling operations that employ floating or sea-based platforms and rigs, without departing from the scope of the disclosure.

As illustrated, the drilling assembly 100 may include a drilling platform 102 that supports a derrick 104 having a traveling block 106 for raising and lowering a drill string 108. The drill string 108 may include, but is not limited to, drill pipe and coiled tubing, as generally known to those skilled in the art. A kelly 110 may support the drill string 108 as it is lowered through a rotary table 112. A drill bit 114 may be attached to the distal end of the drill string 108 and may be driven either by a downhole motor and/or via rotation of the drill string 108 from the well surface. The drill bit 114 may include, but is not limited to, roller cone bits, PDC bits, natural diamond bits, any hole openers, reamers, coring bits, etc. As the drill bit 114 rotates, it may create a wellbore 116 that penetrates various subterranean formations 118. In an embodiment, the drill bit 114 may penetrate reservoir section 136.

Drilling fluid 122 comprises an aqueous or non-aqueous base fluid and a halide. A pump 120 (e.g., a mud pump) may circulate drilling fluid 122 through a feed pipe 124 and to the kelly 110, which conveys the drilling fluid 122 downhole through the interior of the drill string 108 and through one or more orifices in the drill bit 114 and into reservoir section 136. The drilling fluid 122 may then be circulated back to the surface via an annulus 126 defined between the drill string 108 and the walls of the wellbore 116. At the surface, the recirculated or spent drilling fluid 122 may exit the annulus 126 and may be conveyed to one or more fluid processing unit(s) 128 via an interconnecting flow line 130. The fluid processing unit(s) 128 may include, but is not limited to, one or more of a shaker (e.g., shale shaker), a centrifuge, a hydrocyclone, a separator (including magnetic and electrical separators), a desilter, a desander, a separator, a filter (e.g., diatomaceous earth filters), a heat exchanger, and/or any fluid reclamation equipment. The fluid processing unit(s) 128 may further include one or more sensors, gauges, pumps, compressors, and the like used store, monitor, regulate, and/or recondition the drilling fluid 122. The sample of the drilling fluid 122 may be obtained before or after the drilling fluid is processed by the fluid processing unit(s) 128.

After passing through the fluid processing unit(s) 128, a “cleaned” drilling fluid 122 may be deposited into a nearby retention pit 132 (i.e., a mud pit). While illustrated as being arranged at the outlet of the wellbore 116 via the annulus 126, those skilled in the art will readily appreciate that the fluid processing unit(s) 128 may be arranged at any other location in the drilling assembly 100 to facilitate its proper function, without departing from the scope of the scope of the disclosure. One or more drilling fluid additives may be added to the drilling fluid 122 via a mixing hopper 134 communicably coupled to or otherwise in fluid communication with the retention pit 132. In some examples, the drilling fluid additives may comprise a halide that may be added to the drilling fluid 122 via the mixing hopper. The mixing hopper 134 may include, but is not limited to, mixers and related mixing equipment known to those skilled in the art. Alternatively, the drilling fluid additives may be added to the drilling fluid 122 at any other location in the drilling assembly 100. While FIG. 1 shows only a single retention pit 132, there could be more than one retention pit 132, such as multiple retention pits 132 in series. Moreover, the retention pit 132 may be representative of one or more fluid storage facilities and/or units where the drilling fluid additives may be stored, reconditioned, and/or regulated until added to the drilling fluid 122.

It should be clearly understood that the example system illustrated by FIG. 1 is merely a general application of the principles of this disclosure in practice, and a wide variety of other examples are possible. Therefore, the scope of this disclosure is not limited in any manner to the details of FIG. 1 as described herein.

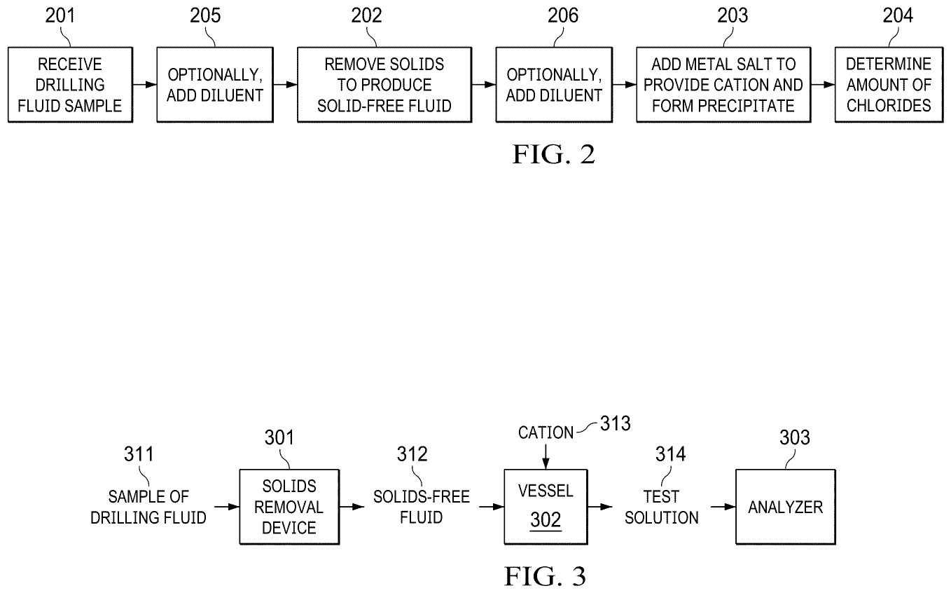

FIG. 2 is a diagram illustrating a method to determine the halide concentration of a drilling fluid sample according to an example of the present disclosure. In block 201, a sample of a drilling fluid is received. In block 202, the solids can be removed from the sample to produce a substantially solids-free fluid. The solids can be removed from the drilling fluid sample using any suitable device and method. Examples of suitable devices and methods include filtration, centrifugation, simple settling, or dissolution. Testing may be conducted on at least a portion of the substantially solids-free fluid. Optionally, the drilling fluid sample may be diluted. Dilution of the drilling fluid sample may be performed at block 205 and/or at block 206, which is before and/or after the solids are removed to produce the substantially solids-free fluid. If the drilling fluid is a non-aqueous fluid, the dilution step is not optional and the sample is diluted with a solution of water and an organic solvent as described above.

At block 203, at least a portion of the substantially solids-free fluid fluid may be contacted with a suitable metal salt to provide a cation of Ag⁺, Pb²⁺, Hg²⁺, or Cu⁺ to form a precipitate in the test solution. For block 204, the level of halide precipitate in the test solution can be measured and compared to known values of turbidity and concentration of halide. The halide precipitate can be measured using methods known to those in the art. Examples of analysis include turbidity, volumetric analysis, and gravimetric weight analysis.

It should be clearly understood that the example system illustrated by FIG. 2 is merely a general application of the principles of this disclosure in practice, and a wide variety of other examples are possible. Therefore, the scope of this disclosure is not limited in any manner to the details of FIG. 2 as described herein.

FIG. 3 is a flow chart showing use of a system for measuring the halide concentration of a drilling fluid sample. A solids removal device 301 may remove solids from a sample of a drilling fluid 311 to produce a substantially solids-free fluid 312. A vessel 302 is used to contain the substantially solids-free fluid and receive a metal salt sufficient for providing the cation. The test solution 314 is produced from the ion exchange of the halide and metal salt. An analyzer 303 may determine the concentration of the halide within the test solution 314. Examples of a solids removal device include a filter and a centrifuge. In some examples, the analyzer 303 may be a turbidimeter, a mass balance, a graduated volumetric container (e.g., a graduated centrifuge tube or a graduated cylinder). It is to be understood that an analyzer is any equipment which may be used to perform an analysis of the sample and/or its contents such as measuring turbidity, viewing the volume of precipitate, or weighing the mass of the precipitate.

It should be clearly understood that the example system illustrated by FIG. 3 is merely a general application of the principles of this disclosure in practice, and a wide variety of other examples are possible. Therefore, the scope of this disclosure is not limited in any manner to the details of FIG. 3 as described herein.

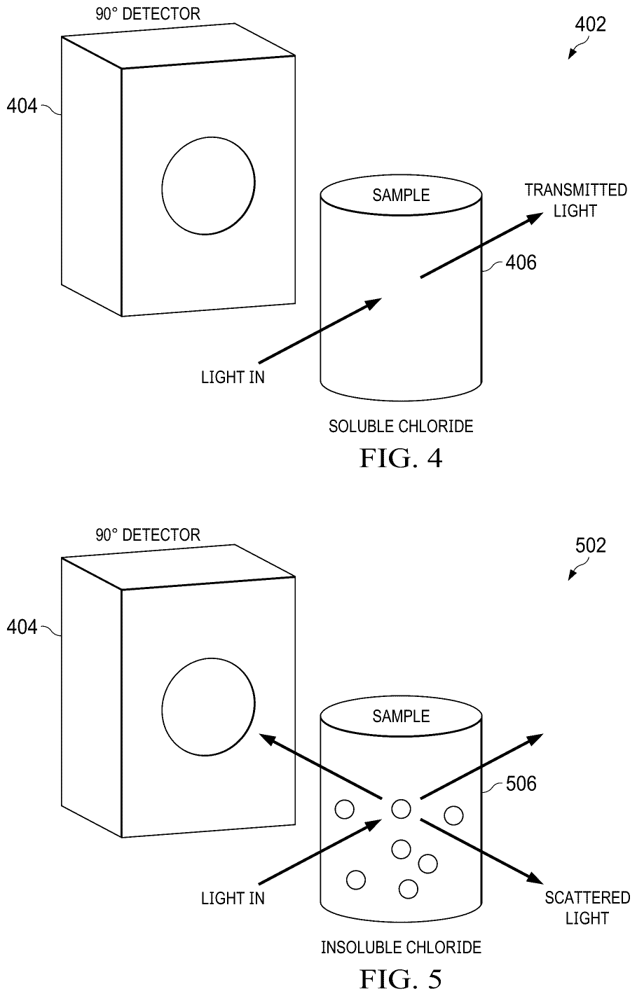

FIG. 4 is a diagram showing the use of a turbidimeter to determine the halide concentration of the drilling fluid sample 406. At setup 402, a detector 404 is positioned at a 90° angle from a light source transmitting light through the drilling fluid sample 406. In the setup of 402, the halide is soluble and the light is not scattered.

FIG. 5 is a diagram showing the use of a turbidimeter to determine the halide concentration of the drilling fluid sample 506. At setup 502, a detector 404 is positioned at a 90° angle from a light source transmitting light through the drilling fluid sample 506. In the image of 402, the halide is insoluble and scatters the emitted light as it passes through drilling fluid sample 506. The detector 404 is able to measure the scattered light. The measurement of the scattered light is analyzed and used to determine a concentration for the halide concentration in the drilling fluid sample.

It should be clearly understood that the example system illustrated by FIGS. 4 and 5 is merely a general application of the principles of this disclosure in practice, and a wide variety of other examples are possible. Therefore, the scope of this disclosure is not limited in any manner to the details of FIGS. 4 and 5 as described herein.

To facilitate a better understanding of the present embodiments, the following examples of certain aspects of some embodiments are given. In no way should the following examples be read to limit, or define, the entire scope of the embodiments.

### Examples

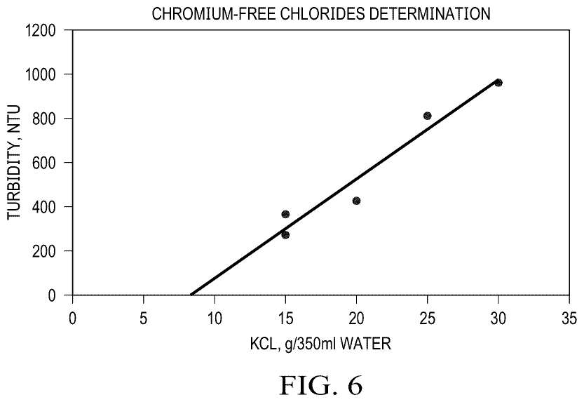

An experiment was performed to analyze the relationship between increasing halide concentration and turbidity. Water containing potassium halide at different concentrations was treated with silver nitrate (0.0282 N). For each sample, one mL of each fluid was added to a vial. Ten mL of DI water was added and the vial shaken. A 0.5 mL aliquot of this solution was measured and transferred to a new vial. Two mL of silver nitrate was added which caused a precipitate to form. Eight mL of DI water was added to the vial. The suspensions were then analyzed with a turbidimeter. Turbidity was shown to increase with increasing potassium halide content. The results are provided in Table 1 below and illustrated by the graph presented in FIG. 6.

TABLE 1

| Fluids for Testing | | Result |
|---|---|---|
| Water, g | KCl, g | NTU |
| 350 | 15 | 273 |
| 350 | 15 | 364 |
| 350 | 20 | 429 |
| 350 | 25 | 818 |
| 350 | 30 | 955 |

The systems disclosed herein may directly or indirectly affect one or more components or pieces of equipment associated with or which may come into contact with the safety valves disclosed herein such as, but not limited to, wellbore casing, wellbore liner, completion string, insert strings, drill string, coiled tubing, slickline, wireline, drill pipe, drill collars, mud motors, downhole motors and/or pumps, cement pumps, surface-mounted motors and/or pumps, centralizers, turbolizers, scratchers, floats (e.g., shoes, collars, valves, etc.), logging tools and related telemetry equipment, actuators (e.g., electromechanical devices, hydromechanical devices, etc.), sliding sleeves, production sleeves, plugs, screens, filters, flow control devices (e.g., inflow control devices, autonomous inflow control devices, outflow control devices, etc.), couplings (e.g., electro-hydraulic wet connect, dry connect, inductive coupler, etc.), control lines (e.g., electrical, fiber optic, hydraulic, etc.), surveillance lines, drill bits and reamers, sensors or distributed sensors, downhole heat exchangers, valves and corresponding actuation devices, tool seals, packers, cement plugs, bridge plugs, and other wellbore isolation devices, or components, and the like.

Provided is an apparatus for determining a halide concentration in a sample of a drilling fluid in accordance with the disclosure and the illustrated FIGs. An example apparatus comprises an analyzer configured to determine an amount of a halide precipitate in a substantially solids-free sample of a drilling fluid by measuring the amount of the precipitated halide produced upon contacting the substantially solids-free sample of drilling fluid with a cation.

Additionally or alternatively, the apparatus may include one or more of the following features individually or in combination. The cation may comprise Ag⁺, Pb²⁺, Hg²⁺, Cu⁺, or a combination thereof. The halide precipitate may comprise silver halide, lead halide, mercury halide, copper halide, or a combination thereof. The analyzer may not use chromium or chromium salts. The analyzer may not use titration. The drilling fluid may be an aqueous-based drilling fluid. The drilling fluid may be a non-aqueous-based drilling fluid.

Provided are methods for determining a halide concentration in a sample of a drilling fluid in accordance with the disclosure and the illustrated FIGs. An example method comprises receiving a sample of a drilling fluid, removing solids from the sample of the drilling fluid to produce a substantially solids-free fluid, contacting the substantially solids-free fluid with a cation to produce a halide precipitate in a test solution, and determining an amount of halide within the drilling fluid sample by measuring an amount of the halide precipitate in the test solution.

Additionally or alternatively, the method may include one or more of the following features individually or in combination. Measuring the amount of the precipitate may comprise performing turbidity analysis of the test solution, performing gravimetric weight analysis of the precipitate, or performing volumetric analysis of the precipitate. The cation may comprise Ag⁺, Pb²⁺, Hg²⁺, Cu⁺, or a combination thereof. The halide precipitate may comprise silver halide, lead halide, mercury halide, copper halide, or a combination thereof. The analyzer may not use chromium or chromium salts. The analyzer may not use titration. The drilling fluid may be an aqueous-based drilling fluid. The drilling fluid may be a non-aqueous-based drilling fluid. The method may further comprise diluting the drilling fluid sample with an organic solvent and/or an aqueous fluid prior to the removing solids from the sample to produce a substantially solids-free fluid thereby producing a diluted drilling fluid sample. Removing solids from the sample to produce a substantially solids-free fluid may further comprise filtering the sample, centrifuging the sample, allowing the solids to settle and then be removed, or dissolution of the solids. The method may further comprise not adjusting a concentration of the halide in the drilling fluid based on a measured amount of the halide precipitate in the test solution. The method may further comprise adjusting a concentration of the halide in the drilling fluid based on a measured amount of the halide precipitate in the test solution.

Provided are systems for determining a halide concentration in a sample of a drilling fluid in accordance with the disclosure and the illustrated FIGs. An example system comprises a solids removal device configured to remove solids from a sample of a drilling fluid to produce a substantially solids-free fluid, a vessel configured to receive at least a portion of the substantially solids-free fluid and a cation and also to collect a halide precipitate produced upon contacting the substantially solids-free fluid with the cation, and an analyzer configured to determine an amount of the halide precipitate within the substantially solids-free fluid by measuring an amount of the halide precipitate.

Additionally or alternatively, the system may include one or more of the following features individually or in combination. The analyzer may be a turbidimeter. The solids removal device may comprise at least one of a filter, a centrifuge, a vessel configured to allow the solids to settle and be removed, or a vessel comprising an acid configured to dissolve the solids. The analyzer may be a graduated volumetric container and the amount of the halide is determined by measuring the volume of the precipitate in the graduated volumetric container. The precipitate may be dried and weighed and the analyzer comprises a scale to determine a weight of the precipitate. The cation may comprise Ag⁺, Pb²⁺, Hg²⁺, Cu⁺, or a combination thereof. The halide precipitate may comprise silver halide, lead halide, mercury halide, copper halide, or a combination thereof. The analyzer may not use chromium or chromium salts. The analyzer may not use titration. The drilling fluid may be an aqueous-based drilling fluid. The drilling fluid may be a non-aqueous-based drilling fluid.

The preceding description provides various examples of the systems and methods of use disclosed herein which may contain different method steps and alternative combinations of components. It should be understood that, although individual examples may be discussed herein, the present disclosure covers all combinations of the disclosed examples, including, without limitation, the different component combinations, method step combinations, and properties of the system. It should be understood that the compositions and methods are described in terms of “comprising,” “containing,” or “including” various components or steps. The systems and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as ranges from any lower limit may be combined with any other lower limit to recite a range not explicitly recited. In the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

One or more illustrative examples incorporating the examples disclosed herein are presented. Not all features of a physical implementation are described or shown in this application for the sake of clarity. Therefore, the disclosed systems and methods are well adapted to attain the ends and advantages mentioned, as well as those that are inherent therein. The particular examples disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown other than as described in the claims below. It is therefore evident that the particular illustrative examples disclosed above may be altered, combined, or modified, and all such variations are considered within the scope of the present disclosure. The systems and methods illustratively disclosed herein may suitably be practiced in the absence of any element that is not specifically disclosed herein and/or any optional element disclosed herein.

Although the present disclosure and its advantages have been described in detail, it should be understood that various changes, substitutions and alterations can be made herein without departing from the spirit and scope of the disclosure as defined by the following claims.

What is claimed is:

1. A method for determining the halide concentration in a drilling fluid, the method comprises:
receiving a sample of a drilling fluid,
removing solids from the sample of the drilling fluid to produce a substantially solids-free fluid,
contacting the substantially solids-free fluid with a cation to produce a halide precipitate in a test solution, and
determining an amount of halide within the drilling fluid sample by measuring an amount of the halide precipitate in the test solution.

2. The method of claim 1, wherein the amount of precipitate may be measured by turbidity analysis of the test solution, by gravimetric weight analysis of the precipitate, or by volumetric analysis of the precipitate.

3. The method of claim 1, wherein the drilling fluid is an aqueous-based drilling fluid.

4. The method of claim 1, wherein the drilling fluid is a non-aqueous-based drilling fluid.

5. The method of claim 1, further comprising diluting the drilling fluid sample with an organic solvent and/or an aqueous fluid prior to the removing solids from the sample to produce a substantially solids-free fluid thereby producing a diluted drilling fluid sample.

6. The method of claim 1, wherein the removing solids from the sample to produce a substantially solids-free fluid further comprises filtering the sample, centrifuging the sample, allowing the solids to settle and then be removed, or dissolution of the solids.

7. The method of claim 1, further comprising not adjusting a concentration of the halide in the drilling fluid based on a measured amount of the halide precipitate in the test solution.

8. The method of claim 1, further comprising adjusting a concentration of the halide in the drilling fluid based on a measured amount of the halide precipitate in the test solution.

9. An apparatus comprising:
an analyzer configured to determine an amount of a halide precipitate in a substantially solids-free sample of a drilling fluid by measuring the amount of the precipitated halide produced upon contacting the substantially solids-free sample of drilling fluid with a cation.

10. The apparatus of claim 9, wherein the cation comprises Ag⁺, Pb²⁺, Hg²⁺, Cu⁺, or a combination thereof.

11. The apparatus of claim 9, wherein the halide precipitate comprises silver halide, lead halide, mercury halide, copper halide, or a combination thereof.

12. The apparatus of claim 9, wherein the analyzer does not use chromium or chromium salts.

13. The apparatus of claim 9, wherein the analyzer does not use titration.

14. The apparatus of claim 9, wherein the drilling fluid is an aqueous-based drilling fluid.

15. The apparatus of claim 9, wherein the drilling fluid is a non-aqueous-based drilling fluid.

16. A system comprising:
a solids removal device configured to remove solids from a sample of a drilling fluid to produce a substantially solids-free fluid,
a vessel configured to receive at least a portion of the substantially solids-free fluid and a cation and also to collect a halide precipitate produced upon contacting the substantially solids-free fluid with the cation, and
an analyzer configured to determine an amount of the halide precipitate within the substantially solids-free fluid by measuring an amount of the halide precipitate.

17. The system of claim 16, wherein the analyzer is a turbidimeter.

18. The system of claim 16, wherein the solids removal device comprises at least one of a filter, a centrifuge, a vessel configured to allow the solids to settle and be removed, or a vessel comprising an acid configured to dissolve the solids.

19. The system of claim 16, wherein the analyzer is a graduated volumetric container and the amount of the halide is determined by measuring the volume of the precipitate in the graduated volumetric container.

20. The system of claim 16, wherein the precipitate is dried and weighed and the analyzer comprises a scale to determine a weight of the precipitate.
